(19) United States
(12) Patent Application Publication
(10) Pub. No.: US 2017/0058361 A1
(43) Pub. Date: Mar. 2, 2017

(54) Polypeptides and Antibodies for Assessing Predisposition for Myelodysplastic Syndromes or Myelogenous Tumor, and Method for Screening Therapeutic Drugs Therefor

(71) Applicant: The University of Tokyo, Tokyo (JP)

(72) Inventors: Seishi Ogawa, Tokyo (JP); Masashi Sanada, Tokyo (JP); Kenichi Yoshida, Tokyo (JP)

(21) Appl. No.: 15/353,395

(22) Filed: Nov. 16, 2016

Related U.S. Application Data
(62) Division of application No. 14/236,276, filed on May 27, 2014, now abandoned.

(30) Foreign Application Priority Data
Aug. 2, 2011 (JP) 2011-169662

(51) Int. Cl. C12Q 1/68; C07K 16/18; C07K 14/47

(52) U.S. Cl. CPC C12Q 1/6886; C07K 14/47; C07K 16/18; C12Q 2600/158; C12Q 2600/136; C12Q 2600/156

(57) ABSTRACT

The present invention provides polypeptide and antibody for assessing predisposition of myelodysplastic syndrome or myeloid tumor, and method for screening therapeutic drugs therefor. The polypeptide comprises at least a portion of the U2AF35 gene, at least a portion of the ZRSR2 gene, at least a portion of the SFRS2 gene, or at least a portion of the SF3B1 gene, and is able to serve as a marker for evaluating predisposition for myelodysplastic syndromes or a myelogenous tumor.

### CROSS-REFERENCE TO RELATED APPLICATIONS

This application is a divisional application and claims the benefit under 35 U.S.C. §§120 and 121 of U.S. application Ser. No. 14/236,276 filed on May 27, 2014, which is a US national phase of PCT/JP2012/069711, filed on Aug. 2, 2012, which claims priority to JP 2011-169662, filed on Aug. 2, 2011, the contents of all of which are incorporated herein by reference in their entirety.

### TECHNICAL FILED OF THE INVENTION

The present invention relates to a method of evaluating predisposition for myelodysplastic syndromes or a myelogenous tumor, and a polypeptide and antibody therefor as well as a method of screening for a candidate therapeutic agent or a candidate prophylactic agent for myelodysplastic syndromes or a myelogenous tumor.

### BACKGROUND OF THE INVENTION

Myelodysplastic syndromes (MDS), which is a disorder where erythrocytes, leucocytes and platelets are decreased in peripheral blood although hematopoietic cells are produced in bone marrow, are thought to be caused by oncogenic transformation at the stem cell level because it often progresses to acute leukemia. Currently, the syndrome is classified as refractory anemia (RA), refractory anemia with ringed sideroblasts (RARS), refractory cytopenia with multikineage dysplasia (RCMD), refractory anemia with multikineage dysplasia with ringed sideroblasts (RCMD-RS), refractory anemia with excess blasts (RAEB), 5q-syndrome and the like. In particular, acute myelogenous leukemia (AML) is the most important complication, but a process in which myelodysplastic syndromes progresses to leukemia has not been known.

A myelogenous tumor, which is characterized in that it may progress to uncontrolled production of dysplastic blood cells and acute myelogenous leukemia, is also an associated disorder of myelodysplastic syndromes.

In order to prevent the progress of myelodysplastic syndromes or a myelogenous tumor to acute myelogenous leukemia, early diagnosis and early treatment are required. However, conveniently, only a blood test which may be used only for confirmed diagnosis of the disorders has been available.

Since a clinical course is indolent before leukemic transformation and ineffective hematopoiesis which are symptoms of myelodysplastic syndromes occur, an onset different from that involved in primary acute myelogenous leukemia is suggested. Nonetheless, the genetic basis of these disorders has not fully elucidated. Some gene mutations and cytogenetic changes are involved in the onset, and RAS, TP53, RANX1, ASXL1, c-CBL, IDH1/2, TET2 and EZH2 are included in the gene targets in which a mutation is most frequently found in myeloid dysplasia. However, for example, because known genetic alterations are not found in about 20% of the cases, a mutation in this set of the genes can not fully explain the onset. Interesting insights for this problem have been provided from the research results about haploinsufficiency in RPS14 and miR-145/146 associated with 5q-syndrome. However, in particular, a genetic alteration causing a metaplasia phenotype thereof has not been sufficiently understood.

Meanwhile, the recent development of massively parallel sequencing technology has provided a new opportunity for probing a genetic alteration across the entire genome or the entire protein coding sequences in human cancer at one nucleotide level.

### CITATION LIST

Nonpatent Literature 1: Wahl, M. C., Will, C. L. & Luhrmann, R. The spliceosome design principles of a dynamic RNP machine. Cell 136, 701-718 (2009)

Nonpatent Literature 2: Tronchere, H., Wang, J. & Fu, X. D. A proteinrelatedto splicing factor U2AF35 that interests with U2AF65 and SR proteins insplicing of pre-mRNA. Nature 388, 397-400(1997)

Nonpatent Literature 3: Calvo, S. E., et al. High-throughput, pooled sequencingidentifies mutations in NUBPL and FOXRED1 in human complex I deficiency. NatGenet 42, 851-858 (2010)

Nonpatent Literature 4: Bevilacqua, L., et al. Apopulation-specific HTR2B stop codon predisposes to severe impulsivity. Nature 468, 1061-1066 (2010)

Nonpatent Literature 5: Chen. M. & Manley, J. L. Mechanisms of alternative splicing regulation insights frommolecular and genomics approaches. Nat Rev Mol Cell Biol 10, 741-754(2009)

Nonpatent Literature 6: Subramanian, A., et al. Gene set enrichment analysis a knowledge-based approach for interpretinggenome-wide expression profiles. Proc Natl Acad Sci USA 102, 15545-15550 (2005)

Nonpatent Literature 7: Bhuvanagiri, M., Schlitter, A. M, Hentze, M. W. & Kulozik, A. E. NMD RNA biology meets human geneticmedicine. Biochem J430, 365-377 (2010)

Nonpatent Literature 8: Maquat, L. E. Nonsense-mediatedmRNA decay splicing, translation and mRNP dynamics. NatRev Mol Cell Biol 5, 89-99 (2004)

Nonpatent Literature 9: Shen, H., Zheng, X., Luecke, S. & Green, M. R. The U2AF35-related protein Urpcontacts the 3′splice site to promote U12-type intron splicing and the secondstep of U2-type intron splicing. Genes Dev24, 2389-2394 (2010)

### BRIEF SUMMARY OF THE INVENTION

### Technical Problem

Accordingly, an object of the present invention is to provide, based on genetic diagnosis using the massively parallel sequencing technology, a method of evaluating predisposition for myelodysplastic syndromes or a myelogenous tumor, and a polypeptide and antibody therefor as well as a method of screening for a candidate therapeutic agent or a candidate prophylactic agent for myelodysplastic syndromes or a myelogenous tumor.

### Solution To Problem

The method of evaluating predisposition for myelodysplastic syndromes and a myelogenous tumor according to the present invention is a method of evaluating whether a subject has predisposition for possible development of myelodysplastic syndromes or a myelogenous tumor, the method comprising the step of detecting a gene mutation in at least one gene of the U2AF35 gene (also referred to as U2AF1), the ZRSR2 gene, the SFRS2 gene and the SF3B1 gene using a sample containing human genes of the subject.

Here, in a case where at least one of the following mutations: a substitution of S with F or Y at an amino acid residue at position 34 of a protein translated from the U2AF35 gene, a substitution of Q with R or P at an amino acid residue at position 157 of the protein translated from the U2AF35 gene, any inactivating mutation in a protein translated from the ZRSR2 gene, a substitution of P with H or L or R at an amino acid residue at position 95 of a protein translated from the SFRS2 gene, a substitution of K with E at an amino acid residue at position 700, a substitution of E with D at an amino acid residue at position 622, a substitution of H with Q or D at an amino acid residue at position 662, a substitution of K with N or T or E or R at an amino acid residue at position 666 of a protein translated from the SF3B1 gene are detected, a subject is evaluated as having predisposition for possible development of myelodysplastic syndromes or a myelogenous tumor, and conversely, in a case where not detected, the subject may be indirectly evaluated as not likely having the predisposition.

In order to detect an amino acid substitution described above, a mutation in a gene corresponding to the amino acid substitution may be detected, or a mutation in a protein and a polypeptide translated therefrom may be detected.

Note that as amino acid abbreviations, S represents Ser (serine), F represents Phe (phenylalanine), Y represents Tyr (tyrosine), Q represents Gln (glutamine), R represents Arg (arginine), P represents Pro (proline), E represents Glu (glutamic acid), X represents Xaa (unknown or other amino acids), H represents His (histidine), L represents Leu (leucine), R represents Arg (arginine), K represents Lys (lysine), D represents Asp (aspartic acid), N represents Asn (asparagine) and T represents Thr (threonine).

A polypeptide according to the present invention comprises at least a portion of the U2AF35 gene, and has at least one of the substitution of S with F or Y at an amino acid residue at position 34 or the substitution of Q with R or P at an amino acid residue at position 157, the polypeptide being able to serve as a marker for evaluating predisposition for myelodysplastic syndromes or a myelogenous tumor.

Similarly, a polypeptide according to the present invention may comprise at least a portion of the ZRSR2 gene, and has any inactivating amino acid mutation, the polypeptide being able to serve as a marker for evaluating predisposition for myelodysplastic syndromes or a myelogenous tumor.

A polypeptide according to the present invention may comprise at least a portion of the SFRS2 gene, and has at least one of the substitutions of P with H or L or R at an amino acid residue at position 95, the polypeptide being able to serve as a marker for evaluating predisposition for myelodysplastic syndromes or a myelogenous tumor.

A polypeptide according to the present invention may comprise at least a portion of the SF3B1 gene, and has at least one of the substitution of K with Eat an amino acid residue at position 700, the substitution of E with D at an amino acid residue at position 622, the substitution of H with Q or D at an amino acid residue at position 662, the substitution of K with N or T or E or R at an amino acid residue at position 666, the polypeptide being able to serve as a marker for evaluating predisposition for myelodysplastic syndromes or a myelogenous tumor.

A polypeptide according to the present invention may comprise an amino acid sequence in which one or several amino acids are deleted, substituted or added in any of the above polypeptides, the polypeptide functioning as an antigen against an antibody, the antibody recognizing one of the original polypeptides.

An antibody according to the present invention is characterized by recognizing one of the above polypeptides.

The method of screening for a candidate therapeutic agent or a candidate prophylactic agent for myelodysplastic syndromes and a myelogenous tumor according to the present invention is a method of screening for a pharmaceutical agent for myelodysplastic syndromes or a myelogenous tumor, the method comprising the steps of evaluating whether a test substance can inhibit an expression or an activity of a protein translated from at least one gene of the U2AF35 gene, the ZRSR2 gene, the SFRS2 gene and the SF3B1 gene using a sample containing human genes of a subject, and selecting the test substance capable of inhibiting the expression or the activity of the protein translated from said at least one gene as an effective substance for preventing or treating a state or a disease resulted from myelodysplastic syndromes or a myelogenous tumor.

### Advantageous Effects Of Invention

The present invention can provide simple and accurate diagnosis for myelodysplastic syndromes and a myelogenous tumor by using genetic diagnosis, contributing to early treatment and prevention. Further, the present invention can also be useful for pathology classification and therapy selection.

### BRIEF DESCRIPTION OF THE DRAWINGS

FIG. 1 shows a table indicating gene mutations which repeatedly appear in 32 cases revealed from the whole exon analysis.

FIG. 2 shows (a) a diagram illustrating gene mutations frequency found in the splicing complexes E/A and components thereof, (b) diagrams illustrating mutations in multiple components of the splicing complexes E/A in a myeloid tumor.

FIG. 3 shows graphs indicating (a) frequencies of spliceosome pathway genes, (b) distributions thereof.

FIG. 4 shows (a) a photograph of western blot analysis in which a S34F mutant of U2AF35 was expressed, (b) a graph showing the activation of the NMD pathway thereof, (c) a graph showing the results of qPCR thereof, (d) a graph showing an expression ratio of exons and introns thereof, (e) a graph showing an expression ratio of exons and introns thereof.

FIG. 5 shows (a) a photograph of western blot analysis in which doxycycline inducibility of a mutant and the wild-type U2AF35 was expressed, (b) graphs showing changes in the cell cycle thereof, (c) a graph showing changes in the cell cycles thereof, (d) a graph showing induction of apoptosis thereof, (e) a graph showing functional analysis of a mutant U2AF35 thereof.

### DETAILED DESCRIPTION OF THE INVENTION

Below, embodiments according to the present invention will be described. Prior art documents and conventionally known technologies can be appropriately used in aid of changing designs of the embodiments.

The present inventors have conducted the somatic cell exome analysis using blood samples from 32 cases of myelodysplastic syndromes patients and healthy persons, and have obtained findings that there are abnormalities in the genes involved in RNA splicing and that amino acid substitutions occur therein. Because these gene abnormalities are found in 40% of the patient samples, testing for the gene abnormalities can identify whether a patient has a myeloid abnormality. Further, as a result of investigating 159 cases of myelodysplastic syndromes patients, 88 cases of chronic myelomonocyte leukemia (CMML) patients and the like, similar abnormalities have been found in about 40% of them.

Further, this is the first case which can prove that RNA splicing abnormality may trigger an onset of a disease.

The present inventors have identified mutations in the U2AF35 gene, the ZRSR2 gene, the SFRS2 gene and the SF3B1 gene found in patients of myelodysplastic syndromes or a myelogenous tumor.

Specifically, they are a substitution of S with F or Y at an amino acid residue at position 34 of a protein translated from the U2AF35 gene, a substitution of Q with R or P at an amino acid residue at position 157 of the protein translated from the U2AF35 gene, any inactivating mutation in a protein translated from the ZRSR2 gene, a substitution of P with H or L or R at an amino acid residue at position 95 of a protein translated from the SFRS2 gene, a substitution of K with E at an amino acid residue at position 700, a substitution of E with D at an amino acid residue at position 622, a substitution of H with Q or D at an amino acid residue at position 662, a substitution of K with N or T or E or R at an amino acid residue at position 666 of a protein translated from the SF3B1 gene. Note that for the mutations in the ZRSR2 gene, the substitutions of E with X at an amino acid residue at position 362 and the like are mentioned in Example, but they include any inactivation mutation in a protein translated.

In a case where any of these is detected, a subject is evaluated as having predisposition for possible development of myelodysplastic syndromes or a myelogenous tumor, and conversely, in a case where not detected, the subject may be indirectly evaluated as not likely having the predisposition.

A polypeptide having any of the above gene mutations can be used as a marker for evaluating predisposition for myelodysplastic syndromes or a myelogenous tumor.

Note that a polypeptide refers to a substance in which two or more amino acid residues are connected via a peptide bond, including relatively short peptides or so-called oligopeptides and long chains called proteins. When producing an antibody for detecting a polypeptide, the polypeptide may comprise an amino acid residue other than the genetically encoded 20 amino acid residues or a modified amino acid residue. Modifications therein include acetylation, acylation, ADP-ribosylation, amidation, biotinylation, covalent bonding with a lipid and a lipid derivative, cross-linking formation, a disulfide bond, addition of a carbohydrate chain, addition of a GPI anchor, phosphorylation, prenylation and the like in a main chain having a peptide bond, an amino acid side chain, an amino terminus and a carboxyl terminus.

The polypeptide according to the present invention may further comprise one or several amino acid deletions, substitutions, additions in addition to the amino acid substitutions in the above gene products as long as it can serve as an antigen against an antibody which recognizes the polypeptide. These mutations may be included in a portion other than the epitope which is recognized by an antibody.

These polypeptides can be prepared by directly preparing the peptides by chemical synthesis. In addition, they can also be prepared by producing polynucleotides encoding these by the site directed mutagenesis method and the like, and then expressing them in an appropriate system.

These polypeptides can be prepared by isolating them from a sample such as blood cells taken from a patient of myelodysplastic syndromes or a myelogenous tumor in a case where the polypeptides are included as gene products of polynucleotides in the sample; by chemically synthesizing them based on the information of amino acid sequences including corresponding mutated amino acid residues; by transforming appropriate host cells by the recombinant DNA technology using appropriate polynucleotide-containing vectors and the like, and producing them in the transformed cells; and the like.

Further, a polynucleotide can be isolated by screening a cDNA library prepared from the total mRNA in a sample taken from a patient of myelodysplastic syndromes or a myelogenous tumor using a corresponding polynucleotide as a probe. Further, a polynucleotide can be prepared by introducing a base mutation into the cDNA of the wild type gene based on site directed mutagenesis using a commercially available mutation system and the like.

Such a polynucleotide may be amplified by the PCR (Polymerase Chain Reaction) method, the RT-PCR method, NASBA (Nucleic acid sequence based amplification) method, TMA (Transcription-mediated amplification) method, SDA (Strand Displacement Amplification) method and the like using, for example, genomic DNA or mRNA prepared from a sample such as blood cells obtained from a patient of myelodysplastic syndromes or a myelogenous tumor as a template, and using appropriate primers.

An antibody according to the present invention is characterized by specifically recognizing any of the above polypeptides. The antibody may be a polyclonal antibody or a monoclonal antibody. It is not limited to an antibody having the original intact structure, and may be various derivatives from the intact antibody such as fragments as long as a binding activity is maintained. These antibodies are useful as components for a detection system used for a method of detecting a mutated gene.

Antibody may be produced using a polypeptide or a complex of the polypeptide and a suitable adjuvant as an immunogen. For example, a polyclonal antibody can be obtained from blood serum after immunizing an animal with an immunogen.

A polypeptide can be detected, for example, by using immunologically specific reactions such as EIA and ELISA in which the antibody according to the present invention is used; by amino acid sequencing of a polypeptide such as a gas phase sequencer using the Edman method; by mass spectrometry such as the MALDI-TOF/MS method and the ESIQ-TOF/MS method.

### EXAMPLES

The whole exome sequencing of bone marrow-derived DNA was performed for 32 patients having myelodysplastic syndromes or a related myeloid tumor, using paired CD3 positive T cells or oral mucosa as a germline control. The whole exome approach is a well-established low cost and high performance method for obtaining a comprehensive registry of mutations in protein codes although non-coding mutations and gene rearrangements are undetectable. On average, 79% of the target sequences were analyzed with a depth of more than 20 folds. All candidates (N=509) for a nonsynonymous single nucleotide mutation (SNV) and a small insertion/deletion (indel) were intensively checked by sequencing using the Sanger's method. Finally, 248 somatic mutations (7.8 per sample) including 191 missense mutations, 24 nonsense mutations, 9 splice site mutations and 24 frameshift induced indels were found. Together with a genome copy number profile obtained from the SNP array karyotype analysis, an overview of the myeloid dysplasia genome was obtained from this somatic mutation array.

An unbiased sample set of mutated genes including a majority of the gene targets frequently observed in myeloid dysplasia was used together with a typical genome copy number profile. As expected, mutations in the known gene targets accounted for only 13.3% of all detected mutations (N=33), and the remaining 215 mutations were found in genes where a mutation had not been previously reported for these tumors. However, it was not easy to distinguish a driver event from a passenger event in the latter case. In many cases, a nonsense/frameshift mutation is likely to impair a protein function, and a target thereof will be a candidate for a tumor suppressor gene. Further, inclusion of common genetic pathways which have been implicated in myeloid dysplasia to date would support that they are not passenger events, but play a causative role.

Therefore, mutations in the TP53 related pathway genes (DAPK1, TP53BP1) and the genes involved in chromatin regulation (PHF6, DOT1-L, PHF8) were reasonable candidates for driver mutations. For example, there is a report that a Dot1-L deficient mouse developed severe anemia due to a defective erythrocyte production in the bone marrow. Congenital nonsense mutations in PHF6 (Xq26.3) and PMF8 (Xp11.2) are responsible for X-linked mental retardation syndrome. PHF8 is a member of the histone demethylation enzyme family proteins which are widely accepted as mutation targets frequently observed in human cancers including myeloid dysplasia while loss-of-function mutations in PHF6 have been reported in about 30% of T cell acute lymphoblastic leukemia (ALL).

Further, in the extended case series of Example, 11 mutations in PHF6 were identified in 10 out of 164 cases of myeloid dysplasias. On the other hand, there was a possibility that mutations observed in multiple cases might be interesting driver mutations since they were likely to be frequently present in myeloid dysplasia. Indeed, 9 out of 12 genes in which mutations occurred frequently were included in the most frequent known target, and involved in a critically important gene pathway in myeloid dysplasia.

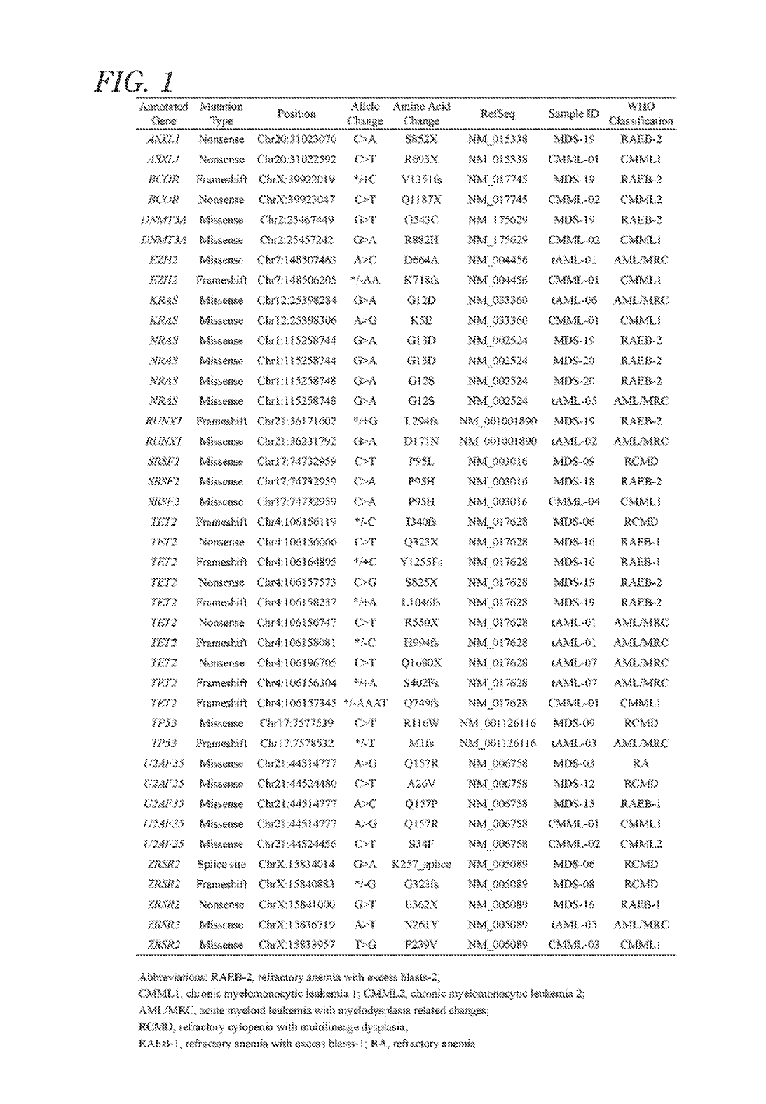

The remaining 3 genes (URAF1, ZRSR2, SFRS2) have not been reported to date, and have belonged to a common pathway known as the RNA splicing machinery, i.e., the spliceosome. Gene mutations which repeatedly appear in the 32 cases reveled by the whole exon analysis are shown in a table of FIG. 1.

Noticeably, when 3 genes (SF3A1, SF3B1, PRPF40B) which showed a mutation in a single case were further included, mutually exclusive mutations were found in 6 components of the splicing machinery in 15 out of 32 cases (47%).

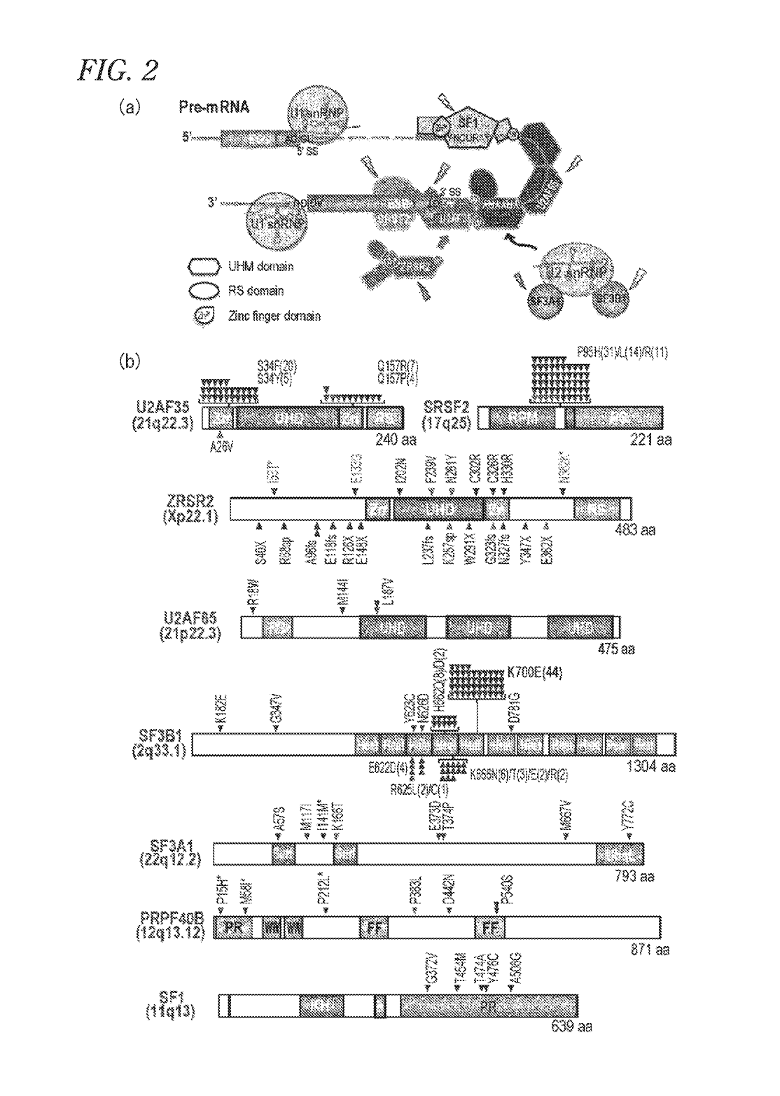

FIG. 2(a) shows a diagram illustrating gene mutations frequency observed in the splicing complexes E/A and components thereof. Arrows indicate mutated components identified by the whole exome sequencing.

In the early stage of RNA splicing, at the same time when U1 snRNP is recruited to the 5′SS of Pre-mRNA during transcription, SF1 and a larger subunit U2AF2 of the U2 auxiliary factor (U2AF) bind to the branch point sequence (BPS) and the polypyrimidine region downstream thereof. A smaller subunit (U2AF35) of U2AF binds to the AG dinucleotide at 3′SS, and interacts with both U2AF2 and a SF protein (for example, SFRS2 (sc35)) through its UHD domain and SR domain, respectively, to form an immediate-early splicing complex (the E complex). ZRSR2, i.e., Urp also interacts with U2AF and a SR protein to play an essential role for RNA splicing. After 3′SS is recognized by these factors, U2 snRNP of the multi-component protein/snRNA complex comprising SF3A1 and SF3B1 is recruited to 3′SS to form the splicing complex A.

In myeloid dysplasia, multiple components of these splicing complexes are affected by gene mutations, resulting in novel and notable examples of pathway mutations in human cancers.

The majority of coding sequences (exons) of metazoan genes are separated by intervening non-coding sequences (introns) which needs to be removed from Pre-mRNA before mature mRNA is produced by a process called RNA splicing. Such functionality is achieved as follows: a set of small molecule nuclear ribonucleoprotein (snRNP) complexes (U1, U2, U4/5/6, U11/12) and many other protein components are recruited on Pre-mRNA in a controlled fashion, reorganized there and then released such that exon-intron boundaries are recognized. Subsequently, two transesterification reactions occur: first between a 5′-splicing site (5′SS) and the branch point sequence (BPS) and then between 5′SS and 3′SS such that the lariat intron is excised out to leave a connected exon.

A point which should be noted with regard to this is a fact that all mutated spliceosome components except for PRPF40B whose function in RNA splicing has not been elucidated are involved in the early stage of RNA splicing. SF1, and the U2 auxiliary factor (U2AF) comprising a U2AF2/U2AF35 heterodimer formed through physical interaction with a SR protein such as SFRS1 or SFRS2 (sc35) are involved in recognition of 3′SS and the adjacent polypyrimidine region. This is thought to be required to later recruit U2 snRNP comprising SF3A1 and SF3B1 to establish the splicing A complex (Nonpatent Literature 1).

Meanwhile, ZRSR2 also known as Urp (an U2AF35 related protein) is another essential component of the splicing machinery. ZRSR2, which has a very similar structure as U2AF35, also physically interacts with U2AF2 as well SFRS1 and SFRS2, and plays a role which is different from that of its homolog U2AF35 (Nonpatent Literature 2).

In order to confirm and expand the initial findings from the whole exome sequencing, the above 6 genes in many myeloid tumors (N=582), and additional 3 spliceosome related genes including U2AF65, SF1, and SRSF1 were investigated for mutations by performing high throughput mutation screening of pooled DNA, and then confirming/identifying candidate mutations (Nonpatent Literatures 3 and 4).

As a result, total 219 mutations were identified in 209 out of 582 myeloid tumor samples by examining 313 tentative positive events from the pooled DNA screening. Mutations in four genes, U2AF35 (N=37), SRSF2 (N=56), ZRSR2 (N=23) and SF3B1 (N=79) accounted for most of the mutations, and mutation rates in SF3A1(N=8), PRPF40B (N=7), U2AF65 (N=4) and SF1 (N=5) were much lower.

FIG. 2(b) shows diagrams illustrating mutations in multiple components of the splicing complexes E/A in myeloid tumors. Arrows indicate mutations in 6 spliceosome components of the splicing complexes E/A while squares indicate known domain structures.

Mutations in U2AF35 and SFRS2 (as well as SF3B1 in some cases) are involved in different hot spots while mutations in ZRSR2 are broadly distributed across its full length, and most of them are nonsense mutations which are responsible for early truncation of a protein or splice site mutations, i.e., indels.

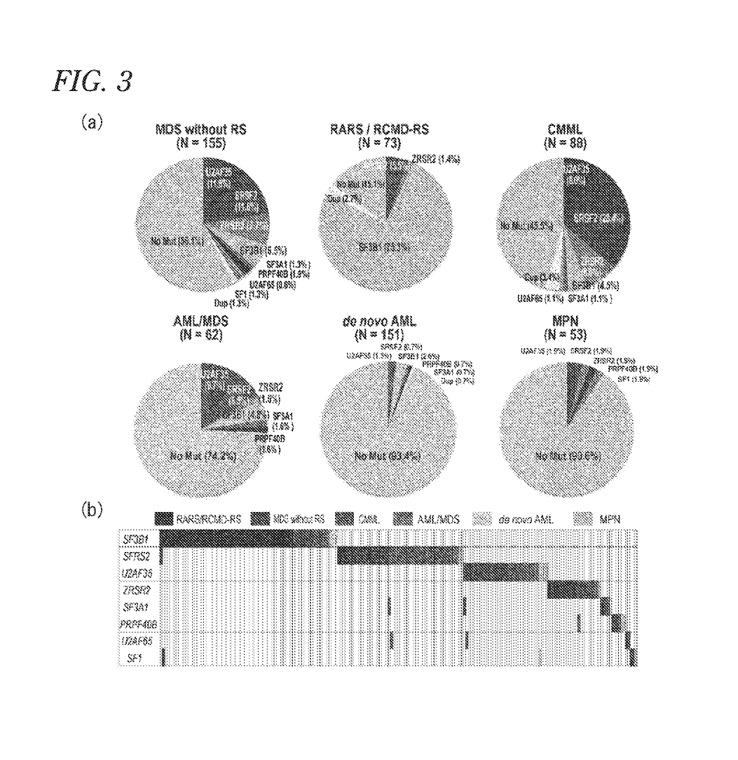

FIG. 3(a) shows frequencies of spliceosome pathway mutations in 513 cases of myeloid tumors including the first 32 cases analyzed by the whole exome study. Frequencies are shown for each tumor type, i.e., MDS, CMML, AML/MDS (including both AML with myeloid dysplasia-related changes and treatment-related AML), primary AML and a myeloproliferative tumor (MPN).

Mutations in the splicing machinery were very specific for diseases showing characteristics of myeloid dysplasia such as MDS with increased ringed sideroblasts (84.9%) or MDS without increased ringed sideroblasts (43.9%), chronic myelomonocytic leukemia (CMML) (54.5%) and treatment-related AML or AML with myeloid dysplasia-related changes (25.8%), but were rare in primary AML (6.6%) and a myeloproliferative tumor (MPN) (9.4%). Further, surprisingly, for refractory anemia with ringed sideroblasts (RARS) and refractory anemia with multikineage dysplasia with ringed sideroblasts (RCMD-RS), many mutations were found in SF3B1, showing 82.6% and 76%, respectively.

FIG. 3(b) shows distributions of two or more spliceosome pathway mutations for these eight genes. Diagnoses of patients are each shown in the top lane.

Mutually exclusive patterns of mutaions in these splicing pathway genes were observed in the large case series, suggesting that different gene mutations in splicing regulation result in a common result.

Meanwhile, frequencies of mutations showed a significant difference across disease types. Surprisingly, SF3B1 mutations were found in the majority of the MDS cases characterized by increased ringed sideroblasts, i.e., refractory anemia with ringed sideroblasts (RARS) (19/23, i.e., 82.6%) and refractory anemia with multikineage dysplasia with ringed sideroblasts (≧15%) (RCMD-RS) (38/50, i.e., 76%) while the mutation frequency in other myeloid tumors was found to be much less. RARS and RCMD-RS account for 4.3% and 12.9% of the MDS cases, respectively. In these disorders, uncontrolled iron metabolism appears to be responsible for refractory anemia. Since the mutation frequency and specificity were very high as described above, SF3B1 mutations were virtually pathognomonic for these MDS subtypes which are characterized by increased ringed sideroblasts, suggesting they are strongly involved in the onset of MDS of these categories. The frequency of SRSF2 mutations was significantly higher in the CMML cases although it was not much striking.

Therefore, different mutations have a common effect on the E/A splicing complex while they may also have a different effect on cell functions, contributing to determining an individual disease phenotype. For example, it has been shown that SRSF2 is also involved in regulation of DNA stability and that decrease in SRSF2 may result in highly frequent DNA mutations. Interestingly in this regard, it was shown that a sample having a SRSF2 mutation significantly had more mutations in other genes as compared with U2AF35 mutations regardless of disease subtypes (p=0.001, multiple linear regression analysis).

Notably, suppose that A26V in one case is a rare exception, U2AF35 mutations were mainly found at two highly conserved amino acid positions (S34 or Q157) within the zinc finger motifs at the N-terminus and the C-terminus adjacent to the UHM domain. SRSF2 mutations were mainly found at P95 within an intervening sequence between the RPM domain and the RS domain. Similarly, SF3B1 mutations were found to be mostly K700E, and to a lesser extent, were found at K666, H662 and E622, which are also conserved across species.

Although the frequently appearing amino acid locations found in these spliceosome genes strongly suggest that these mutations have a gain-of-function property, this is a scenario well proven even for other oncogene mutations including RAS mutations found at codons 12, 13 and 61 as well as V617F, the JAK2 mutation, V600E, the BRAF mutation and recently found Y641, the EZH2 mutation.

In contrast, 23 mutations in ZRSR2 (Xp22.1) were widely distributed across the entire coding region (FIG. 2(b)). Among these, 14 mutations were nonsense mutations or frameshift mutations, or were found in the splicing donor/acceptor sites, which may be responsible for early truncation or a large structural change of a protein which results in loss of function. Together with the fact that the mutations are strongly biased in males (14/14), ZRSR2 most likely serves as a tumor suppressor gene via an X-linked recessive genetic mechanism of action. Remainding 9 ZRSR2 mutations, which were missense mutations, were bound in both males (6 cases) and females (3 cases) while those originated from somatic cells were found only in two cases.

However, these missense nucleotides were not included in either the dbSNP database (Builds 131 and 132) or the 1000 human genome database (calling of snp as of May, 2011). This suggests that many of these missenses SNV, if not all, likely represent functional somatic mutations found particularly in males. Although these mutation hot spots in U2AF35 and SRSF2 were investigated, mutations were not found in ALL (N=24) or lymphoid tumors such as non-Hodgkin's lymphoma (N=87).

All of the gene mutations are specific to MDS, and the mutation frequency in other diseases, especially in myeloid tumors is low. Some cases of AML include those which are progressed from MDS, but the mutation frequency in these gene clusters is significantly higher than that of new AML (de novo AML). This is also effective for distinguishing AML from MDS-derived AML.

Particularly in RARS, the mutation rate in the SF3B1 gene is high, and these gene mutations are essentially determinants of this disease. It has been statistically shown that a group having a mutation in the SRSF2 gene often shows a mutation in other genes. Frequent occurrence of SRSF2 gene mutations is also a characteristic to CMML.

A disease group classified into RCMD according to the current WTO classification can be divided into two groups depending on the presence of SF3B1, SRSF2 gene mutations. The prognosis of a group having these mutations may be better than that of a group having no mutation. Therefore, this is useful for prognostic prediction.

Spliceosome mutations in myeloid dysplasia widely affected the main components of the splicing complexes E/A in a mutually exclusive fashion. This logically suggested that common functional targets of these mutations are exact recognition of an exon-intron boundary, and subsequent recruitment of U2 snRNP to Pre-mRNA via these complexes (Nonpatent Literatures 1, 5).

In order to understand this and in order to obtain an insight about biological/biochemical effects resulted from these splicing mutations, the wild type U2AF35 and the mutant (S34F) U2AF35 were expressed in HeLa cells by the gene transfer method with retrovirus using EGFP as a marker.

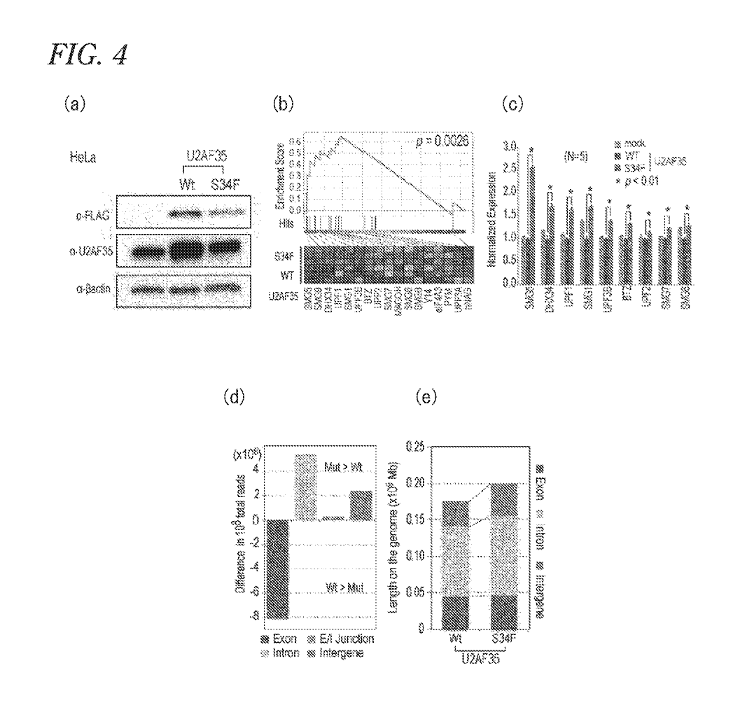

FIG. 4(a) shows a photograph of western-blot-analysis. Shown is the expression of the wild type U2AF35 or the mutant (S34F) U2AF35 introduced in HeLa cells, TF-1 cells used for the gene expression analysis.

GFP positive cells were collected 48 hours after the gene transfer. Then gene expression profiles of the mutant U2AF35 introduced cells and the wild type U2AF35 introduced cells were analyzed using GeneChip® human genome U133 plus 2.0 array analysis and gene set enrichment analysis (GSEA). Subsequently gene set enrichment analysis (GSEA) was performed (Nonpatent Literature 6).

In the present GSEA, all of the expressed genes were first ranked in descending order of differences in gene expression between the wild type U2AF35 introduced cells and the mutant U2AF35 introduced cells. Next a gene set enriched therein was searched. The initial GSEA results revealed several gene sets enriched in the mutant U2AF35 introduced cells. Among these, particularly interesting was a gene set involved in nuclear RNA export (P=0.012). It is because a series of genes (N=6) involved in nonsense-mediated mRNA decay (NMD) were included in the gene set, all of which were involved in core enrichment.

FIG. 4(b) shows a graph illustrating the activation of the NMD pathway resulted from a U2AF35 mutant, and shows the results from the initial GSEA using a gene set of c5.bp.v2.5. symbols (the Gene Ontology). Significant enrichment of a nuclear RNA export gene set in the mutant U2AF35 introduced HeLa cells is shown as compared with the wild type U2AF35 introduced cells.

In the next step, NMD-related genes were selected from the nuclear RNA export gene set to obtain additional NMD-related genes, which gave a more comprehensive NMD pathway gene set. More significant enrichment of the NMD gene set in the mutant U2AF35 introduced HeLa cell is shown. The significance of the gene set was experimentally determined by rearrangement of the 1,000-gene set.

Indeed, this enrichment became even more significant when GSEA was performed in which a more comprehensive NMD gene set was included (P=0.0026) (Nonpatent Literature 7). Microarray expression data were confirmed by quantitative polymerase chain reaction (qPCR).

FIG. 4(c) shows the results from qPCR. Microarray results of the expression of 9 genes which contribute to the core enrichment of the NMD gene set have been obtained. After normalizing against the mean expression (+ S. E.) in the wild type U2AF35 introduced HeLa cells, the mean expression (+ S. E.) of the NMD genes in pseudo introduced HeLa cells, wild type U2AF35 introduced HeLa cells and mutant U2AF35 introduced HeLa cells were plotted. P values were determined by the Mann-Whitney U tests.

Similar enrichment was also observed in a gene expression profile of S34F mutant-introduced TF-1 (a myelodysplastic syndromes-derived cell line in which no spliceosome mutation has been known).

Results from the GSEA of U2AF35 mutant-introduced TF-1 cells showed that the NMD pathway gene set was enriched in U2AF35 introduced cells.

Given that the NMD pathway known as a mRNA quality control mechanism provides a post-transcription mechanism where an abnormal transcription product which prematurely terminates translation is recognized and eliminated (Nonpatent Literature 8), the GSEA results strongly suggested that an abnormal transcription which most likely produces a non-splicing RNA species takes place to induce an NMD activity in mutant U2AF35 introduced cells.

In order to confirm this, a GeneChip® human exon 1.0 ST array (Affymetrix Inc.) was used to perform total transcriptome analysis of these cells. In the analysis, two discrete probe sets showing different levels of evidence that they are exons, i.e., the “core” set (true exon) and the “non-core” set (most likely intron) were individually followed for their behaviors. Results showed that the core set probes and the non-core set probes were differently enriched in probes showing significantly different expression between the wild type introduced cells and the mutant introduced cells (FDR=0.01). The core set probes were significantly enriched in probes significantly downregulated in the mutant U2AF35 introduced cells as compared with wild type U2AF35 introduced cells whereas the non-core set probes were significantly enriched in probes significantly upregulated in the mutant U2AF35 introduced cells. Even when all of the probe sets were included, significantly different enrichments were shown.

Further, the core set probes which showed significant differential expression tended to be upregulated and downregurlated in the wild type U2AF35 introduced cells and the mutant U2AF35 introduced cells, respectively, as compared with the pseudo introduced cells, but the non-core set probes which showed differential expression also showed the same trend.

That is, these exon array results suggested that the wild type U2AF35 correctly promoted true RNA splicing whereas the mutant U2AF35 likely inhibited this process to produce a non-core, resulting in an unspliced intron sequence left behind.

In order to confirm the results from the exon array analysis and in order to show more direct evidence of abnormal splicing in mutant introduced cells, sequencing analysis was performed for mRNA extracted from HeLa cells in which expressions of the wild type U2AF35 and the mutant (S34F) U2AF35 were induced by doxycycline. Differential enrichment of the probe sets was reproduced between the two HeLa samples by directly counting respective readings of the core set probes and the non-core set probes.

Abnormal splicing in the mutant U2AF35 introduced cells was more directly proved by the evaluation of reading counts at various segments, i.e., exons, introns and intergenic regions as well as exon/intron junction regions. First, after adjusted against the total mapped readings, the wild type U2AF35 introduced cells showed increased readings in exon segments, but decreased readings in other segments as compared with the mutant U2AF35 introduced cells. Readings from the mutant introduced cells were more broadly mapped across the genome region as compared with the wild type U2AF35 introduced cells, which could be roughly explained by non-exon readings.

FIG. 4(d) and (e) show results from the studies in which the wild type (normal) U2AF35 and the mutant U2AF35 were expressed in HeLa cells, and RNA sequencing was performed to compare the wild type with the mutant to investigate an expression ratio of exons and introns. Exons, introns and the like in the genomes of respective cells in which the wild type gene or the mutant gene was introduced are shown in FIG. 4(e).

In a case where the wild type gene was introduced, many exons but almost no introns were observed. In contrast, in a case where the mutant gene was introduced, many introns but almost no exons were observed.

Further, when true exon/intron junction regions were included, the number of readings was significantly increased in the mutant U2AF35 introduced cells as compared with in the wild type U2AF35 introduced cells. These results clearly showed that failure in splicing ubiquitously occurred in the mutant U2AF35 introduced cells.

Next, effects of a functional disorder of the E/A splicing complex due to a gene mutation on a phenotype of a hematopoietic cell were studied. First, lentivirus constructs which express the S34F U2AF35 mutant and the wild type U2AF35 under the control of a tetracycline inducible promoter were introduced into TF-1 cells and HeLa cells. After inducing their expression, effects of the U2AF35 mutant on cell proliferation were studied.

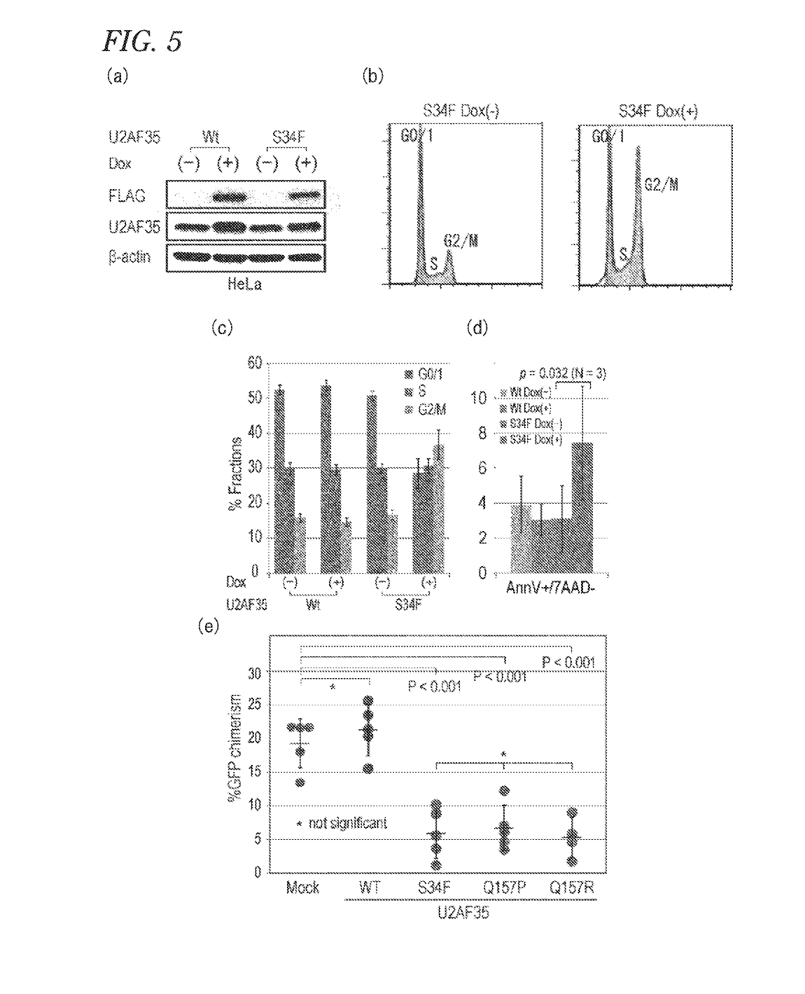

FIG. 5(a) shows a photograph of western blot analysis. It shows doxycycline inducible expression of the mutant (S34F) and the wild type U2AF35 in the HeLa cells and the TF-1 cells 72 hours after induction.

Unexpectedly, after inducing gene expression with doxycycline, the mutant U2AF35 introduced cells showed decreased cell proliferation, which was not observed in the wild type U2AF35 introduced cells.

When examining the functional analysis of the mutant U2AF35, the cell proliferation assays of the HeLa cells and the TF-1 cells showed that growth was significantly suppressed after inducing U2AF35 expression in the mutant U2AF35 introduced cells, but not in the wild type U2AF35 introduced cells.

In order to confirm these observation results in primary cultured cells, either mutant (S34F, Q157P, Q157R) constructs or a wild type U2AF35 construct and a pseudo construct each having an EGFP marker were introduced into a highly purified hematopoietic stem cell population (CD34⁻c-Kit⁺Scal⁺Lin⁻, CD34⁻KSL) prepared from the bone marrow of a C57BL/6 (B6)-Ly5.1 mouse.

Next, the bone marrow reconstitution ability of these gene transfer cells was studied by the competitive bone marrow reconstitution assay. After mixed with total bone marrow cells from a B6-Ly5.1/5.2 F1 mouse, the gene transfer cells were transplanted into a B6-Ly5.2 recipient which had received a lethal dose of radiation. Six weeks later, peripheral blood chimaerism originated from GFP positive cells was evaluated by flow cytometry.

Evaluation of EGFP % in the gene transfer cells and overall proliferation by ex vivo follow-up studies confirmed that each recipient mouse had received the similar number of GFP positive cells among various retrovirus groups.

FIG. 5(b) and (c) show graphs illustrating how the wild type (normal) U2AF35 gene expressed in HeLa cells affects cell cycles. When the mutated gene is expressed, many cells are found to be arrested at G2/M. Further, in a case where the wild type U2AF35 gene is expressed, similar cell cycles are observed even when compared with the control group in which neither of the genes is expressed. This suggests that the cell cycles are arrested at G2/M only in those where the mutated genes are expressed.

FIG. 5(d) shows a graph illustrating that the expression of the wild type (normal) and the mutant U2AF35 in HeLa cells has induced apoptosis. Induction of apoptosis is detected using positive anexyn V and negative 7AAD as indicators. Only the group in which the mutated gene is expressed is found to show apoptosis.

FIG. 5(e) shows the results from the competitive bone marrow reconstitution assay of the CD34⁻KSL cells to which one of the 3 U2AF35 mutants is introduced, as compared with the pseudo introduced cells and the wild type U2AF35 introduced cells. A horizontal line and a vertical line show the mean and S.D. of the results obtained from 5 mice, respectively. Outliers were excluded from the analysis by the Grubbs' outlier test. The significance in differences was determined by the Bonferroni multiple comparison test. The vertical axis shows GFP positive Ly5.1 cell % in peripheral blood after 6 weeks of transplantation.

The wild type U2AF35 introduced cells showed slightly higher bone marrow reconstruction ability than the pseudo introduced cells. In contrast, recipients of the cells to which one of the U2AF35 mutants was introduced showed significantly lower GFP⁺ cell chimaerism than those received the pseudo introduced cells or the wild type U2AF35 introduced cells, showing that the bone marrow reconstruction ability of hematopoietic stem/precursor cells in which a U2AF35 mutant was expressed was impaired. The opposite behaviors of the wild type U2AF35 construct and the mutant U2AF35 construct also suggested that these mutants caused the loss of the U2AF35 function probably through a dominant negative effect against the wild type protein.

The whole exome sequencing studies described above have revealed the complexity of the novel pathway mutations found in about 40 to 60% of myeloid dysplasia patients which affect multiple different components of the splicing machinery (the splicing complexes E/A).

The RNA splicing system is a unique characteristic of metazoan species. In this system, a coding sequence is prepared as multiple fragments which are separately located in the genome DNA. After transcription, intervening sequences are deleted to reconnect them, creating a functional mRNA copy (Nonpatent Literature 1). This appears to be a tedious or wasteful process. However, major protein resources are provided by this, and the functional diversity is provided by alternative splicing in spite of the limited number of the genes (Nonpatent Literature 5).

Accordingly, if the integrity of the whole transcriptome is assured by sophisticatedly adjusting such a complicated process using the spliceosome machinery, we certainly have to pay a price in return for it. Abnormal RNA splicing, i.e., cancer specific alternative splicing is implicated in the development of human cancers including myelodysplastic syndromes and other hematopoietic tumors although the exact mechanism thereof has not been elucidated.

Considering that the present exome sequencing appeared to have a sufficient range and sensitivity for detecting other spliceosome components, it is safe to say that other spliceosome components were not affected although spliceosome mutations were broadly and specifically found in the components involved in the splicing complexes E/A. These mutations were almost completely mutually exclusive and very specific to myeloid dysplasia. This suggested that a functional disorder of these complexes is a common consequence of these mutations, and they are involved in the development of this distinct category of myeloid tumors by disturbing a physiological process of RNA splicing. Although there is no direct evidence to prove that these mutations actually create abnormally spliced mRNA species, the enhanced NMD activity in the mutant U2AF35 introduced cells strongly suggested that the mutant U2AF35 promotes increased production of mRNA species having a premature termination codon.

Meanwhile, the results from the competitive bone marrow reconstitution assay and the in vitro cell proliferation assay can not be easily interpreted. The mutant U2AF35 appeared to suppress cell growth/proliferation instead of providing an advantage for growth and facilitating clonal selection as expected from classical oncogenes. Interestingly, with regard to the observation results, there is a report that apoptosis was induced by the ZRSR2 knockdown in HeLa cells, which supports the common consequence of these pathway mutations (Nonpatent Literature 9). Although there is no clear answer for the apparently contradicting phenotype, this suggests that a pathogenetic role of a U2AF35 mutation needs to be understood in the context of mutations in other genes and/or neoplastic growth of U2AF35 mutant cells or a tumor cell environment compatible with clonal selection.

It should be noted that the most common clinical manifestation of myelodysplastic syndromes is not uncontrolled cell proliferation, but is serious cytopenia in multiple cell lines due to ineffective hematopoiesis accompanied by increased apoptosis. In this regard, the results from the studies about the development 5q-syndrome reporting that apoptosis of an erythrocyte precursor is increased due to RPS14 haploinsufficiency, but myeloid proliferation is not increased can provide a suggestion.

The present discovery of highly frequent mutations in the spliceosome components clearly showed the importance of disordered RNA splicing in the development of myelodysplastic syndromes and related myeloid tumors. Many ZRSR2 mutations cause early truncation of a protein while mutations in U2AF35, SFRS2 and SF3B1 appear to target specific amino acid positions, and appear to be gain-of-function types. Such gain-of-function can be explained by any dominant negative mechanism. These mutations appear to be involved in a common step of RNA splicing. However, there also exits a difference in their distribution among disease types. In CMML, the frequency of SFRS2 mutations is high, which are also often overlapped with other common mutations.

Further, the present invention can provide a method of screening for a candidate therapeutic agent or a candidate prophylactic agent for myelodysplastic syndromes or a myelogenous tumor.

That is, the method comprises the steps of evaluating whether a test substance can inhibit an expression or an activity of a protein translated from at least one gene of the U2AF35 gene, the ZRSR2 gene, the SFRS2 gene and the SF3B1 gene using a sample containing human genes of a subject; and selecting the test substance capable of inhibiting the expression or the activity of the protein translated from said at least one gene as an effective substance for preventing or treating a state or a disease resulted from myelodysplastic syndromes or a myelogenous tumor.

The method may comprise the steps of administering a test substance to an non-human animal; measuring an expression or an activity of at least one gene of the U2AF35 gene, the ZRSR2 gene, the SFRS2 gene and the SF3B1 gene in the non-human animal to which the test substance is administered; and selecting the test substance capable of inhibiting the expression or the activity as an effective substance.

A mutated gene may be noted as a similar method of screening for a candidate therapeutic agent or a candidate prophylactic agent.

That is, the method comprises the steps of evaluating whether a test substance can inhibit an expression or an activity of a protein translated from at least one gene of the U2AF35 gene, the ZRSR2 gene, the SFRS2 gene and the SF3B1 gene using a sample containing human genes of a subject; and selecting the test substance capable of inhibiting the expression or the activity of the protein translated from said at least one gene as an effective substance for preventing or treating a state or a disease resulted from myelodysplastic syndromes or a myelogenous tumor.

The method may comprise the steps of administering a test substance to an non-human animal; determining a mutation in at least one gene of the U2AF35 gene, the ZRSR2 gene, the SFRS2 gene and the SF3B1 gene in the non-human animal to which the test substance is administered; and selecting the test substance capable of decreasing the mutation as an effective substance.

For the mutations in the above genes, a substitution of S with F or Y at an amino acid residue at position 34 of a protein translated from the U2AF35 gene, substitution of Q with R or P at an amino acid residue at position 157 of the protein translated from the U2AF35 gene, any inactivating mutation in a protein translated from the ZRSR2 gene, a substitution of P with H or L or R at an amino acid residue at position 95 of a protein translated from the SFRS2 gene, a substitution of K with E at an amino acid residue at position 700, a substitution of E with D at an amino acid residue at position 622, a substitution of H with Q or D at an amino acid residue at position 662, a substitution of K with N or T or E or R at an amino acid residue at position 666 of a protein translated from the SF3B1 gene may be effectively used.

For the test substances, any known compounds and novel compounds may be used, including, for example, organic small molecule compounds, compound libraries created by the combinatorial chemistry technology, nucleic acids (nucleosides, oligonucleotides, polynucleotides and the like), carbohydrates (monosaccharides, disaccharides, oligosaccharides, polysaccharides and the like), lipids (saturated or unsaturated linear, branched, cyclic fatty acids and the like), amino acids, proteins (oligopeptides, polypeptides and the like), random peptide libraries created by solid phase synthesis and the phage display method, natural compounds from microorganisms, animals and plants, marine organisms and the like.

Non-human animals include, for example, mammals such as mouse, rat, hamster, guinea pig, rabbit, canine, monkey and the like.

In a case where a non-human animal is used, conventionally known methods can be used for administering a test substance to the non-human animal. For example, they include oral administration, parenteral administration (intravenous injection, subcutaneous injection, intraperitoneal injection, local infusion and the like). Dosage, dosing interval, dosing period and the like are to be suitably selected depending on the test substance and the animal to be used.

In order to evaluate the efficacy of a test substance, conventionally known methods may be used. For example, expression levels of proteins translated from the above genes or mutated genes thereof in a non-human animal to which the test substance is administered may be measured.

The expression levels can be measured by, for example, obtaining a biological sample such as bone marrow and blood from a non-human animal, and measuring a transcription product and the like in the sample.

Further, screening may be performed using tissues and cells, and biological materials, for example, bone marrow, blood and the like from a non-human animal.

Cells in which expression levels of proteins translated from the above genes or mutated genes thereof can be directly evaluated are their expression cells while cells in which the expression levels can be indirectly evaluated are those in which reporter assays can be performed for the transcriptional regulatory domains of the above genes or mutated genes thereof.

Cells in which reporter assays can be performed for the transcriptional regulatory domains of the above genes or mutated genes thereof are those having the transcriptional regulatory domains of the above genes or mutated genes thereof, reporter genes operationally linked to the domains.

Further, cells having the above genes or mutated genes thereof or cells in which mutations are forced to be expressed can be used to evaluate whether a subject have predisposition for another disease different from myelodysplastic syndromes or a myelogenous tumor.

### INDUSTRIAL APPLICABILITY

The present invention is effective for early treatment and prevention of myelodysplastic syndromes or a myelogenous tumor, and industrially useful.

What is claimed is:

1. A polypeptide comprising at least a portion of the U2AF35 gene, having at least one of the substitution of S with F or Y at an amino acid residue at position 34 or the substitution of Q with R or P at an amino acid residue at position 157, wherein the polypeptide is able to serve as a marker for evaluating predisposition for myelodysplastic syndromes or a myelogenous tumor.

2. A polypeptide comprising at least a portion of the ZRSR2 gene, having an inactivating amino acid mutation, wherein the polypeptide is able to serve as a marker for evaluating predisposition for myelodysplastic syndromes or a myelogenous tumor.

3. A polypeptide comprising at least a portion of the SFRS2 gene, having at least one of the substitution of P with H or L or R at an amino acid residue at position 95, wherein the polypeptide is able to serve as a marker for evaluating predisposition for myelodysplastic syndromes or a myelogenous tumor.

4. A polypeptide comprising at least a portion of the SF3B1 gene, having at least one of the substitution of K with E at an amino acid residue at position 700, the substitution of E with D at an amino acid residue at position 622, the substitution of H with Q or D at an amino acid residue at position 662, the substitution of K with N or T or E or R at an amino acid residue at position 666, wherein the polypeptide is able to serve as a marker for evaluating predisposition for myelodysplastic syndromes or a myelogenous tumor.

5. A polypeptide functioning as an antigen against an antibody, the antibody recognizing a polypeptide of any one of claims 1 to 4 comprising an amino acid sequence in which one or several amino acids are deleted, substituted or added in the corresponding polypeptide according to any one of claims 1 to 4.

6. An antibody which recognizes a polypeptide of claim 5.

7. An antibody which recognizes a polypeptide of any one of claims 1 to 4.

8. A method of screening for a candidate therapeutic agent or a candidate prophylactic agent for myelodysplastic syndromes or a myelogenous tumor, the method comprising the steps of:
evaluating whether a test substance can inhibit an expression or an activity of a protein translated from at least one gene of the U2AF35 gene, the ZRSR2 gene, the SFRS2 gene and the SF3B1 gene using a sample containing human genes of a subject,
selecting the test substance capable of inhibiting the expression or the activity of the protein translated from said at least one gene as an effective substance for preventing or treating a state or a disease resulted from myelodysplastic syndromes or a myelogenous tumor.
